(19) United States
(12) Patent Application Publication
(10) Pub. No.: US 2021/0265037 A1
(43) Pub. Date: Aug. 26, 2021

(54) VIRTUAL REALITY-BASED COGNITIVE TRAINING SYSTEM FOR RELIEVING DEPRESSION AND INSOMNIA

(71) Applicant: SYinotech, Busan (KR)

(72) Inventor: Yean Hwa LEE, Busan (KR)

(21) Appl. No.: 16/818,514

(22) Filed: Mar. 13, 2020

(30) Foreign Application Priority Data
Feb. 21, 2020 (KR) 10-2020-0021332

(51) Int. Cl. G16H 20/70; G06T 19/00; G06F 3/01

(52) U.S. Cl. CPC G16H 20/70; G06F 3/015; G06F 3/014; G06T 19/006

(57) ABSTRACT

Disclosed herein is a virtual reality (VR)-based cognitive training system for relieving depression and insomnia. The VR-based cognitive training system includes: a kiosk configured to run VR-based content; a VR display unit configured to receive data from the kiosk and to visually implement the VR-based content; a VR glove unit configured to detect a hand motion of a user and to manipulate the VR-based content; a VR chair unit configured to detect a foot motion of a user and to manipulate the VR-based content; and a brainwave-connected unit configured to detect and analyze brainwaves of the user. When the user wears the VR display unit and the VR glove unit, sits on the VR chair unit and does training according to the VR-based content, the brainwave-connected unit detects and analyzes brainwaves of the user during the training, and the kiosk monitors the effects of the training.

### CROSS-REFERENCE TO RELATED APPLICATION

This application claims the benefit of Korean Patent Application No. 10-2020-0021332 filed on Feb. 21, 2020, which is incorporated herein by reference in its entirety.

### BACKGROUND

### 1. Technical Field

The present disclosure relates generally to a cognitive training system, and more particularly to a system for providing cognitive training for the relief of depression and insomnia by using a virtual reality (VR) apparatus.

### 2. Description of the Related Art

Virtual reality (VR) corresponds to the information activity field that makes it possible to indirectly experience situations, which are difficult to directly experience in the real world due to physical and spatial limitations, through interaction with a human sensory system in a virtual environment constructed using a computer. When systems that implement virtual reality provide a virtual environment in which a user's viewpoint and motion are realized, the user's viewpoint or motion are actually changed in virtual space or a human experiences the environment through an entity that performs actions on behalf of the human body in virtual reality space.

Virtual and augmented reality experts and economists predict that the related market will grow from 5.2 billion dollars to 162 billion dollars in 2020. Technology for virtual reality is also expected to be a promising technology that can lead the fourth industrial revolution along with artificial intelligence. In a virtual reality environment, interaction devices have been used to recognize various types of information of users based on input sensor devices. For example, as soon as a ball hits a golf club on a virtual screen, space-aware motion sensors attached to the golf club immediately analyze a user's swing angle and putting posture, the analyzed user information is transmitted directly to a smartphone, and thus the user may correct his or her posture using the information.

However, output devices for generating feedback signals such as reaction force, tactile information, and stimulus information and conveying the information to users in reverse are now in their infancy. Accordingly, there is a considerable growth potential for the development of the technology.

Meanwhile, when VR apparatuses, such as head mount displays (HMDs), are equipped with content helping cognitive training for relieving depression and insomnia, participants may individually take training programs at desired times and places, and thus it is determined that training effects are considerably large.

### RELATED ART DOCUMENTS

### Patent Documents

(Patent document 1) KR101915238 B1

(Patent document 2) KR101944489 B1

(Patent document 3) KR101881986 B1

(Patent document 4) KR101777755 B1

(Patent document 5) KR101828952 B1

(Patent document 6) KR1020120092249 A

### SUMMARY

An object of the present invention is to provide a VR-based cognitive training system for relieving depression and insomnia, which is capable of monitoring training results by detecting and analyzing brainwaves during training when a user does cognitive training by using a VR apparatus.

In order to accomplish the above object, the present invention provides a VR-based cognitive training system for relieving depression and insomnia, the VR-based cognitive training system including: a kiosk configured to run VR-based content for relieving depression and insomnia; a VR display unit configured to receive data from the kiosk and to visually implement the VR-based content; a VR glove unit configured to detect a hand motion of a user and to manipulate the VR-based content; a VR chair unit configured to detect a foot motion of a user and to manipulate the VR-based content; and a brainwave-connected unit configured to detect and analyze brainwaves of the user; wherein when the user wears the VR display unit and the VR glove unit, sits on the VR chair unit and does training according to the VR-based content, the brainwave-connected unit detects and analyzes brainwaves of the user during the training, and the kiosk monitors the effects of the training.

The VR glove unit may include: a hand motion detection module installed at at least one predetermined location and configured to detect a hand motion of the user; a touch detection module installed at at least one predetermined location and configured to recognize a touch; and a tracking detection module configured to detect a trajectory of the VR glove unit.

The VR chair unit may include: a support module configured to be disposed on the ground surface and to form a support; a body module formed to a predetermined length in an upward direction from the center of the support module; a support module coupled to one end of the body module; a saddle module formed at a predetermined location of the body module in a direction perpendicular to the longitudinal direction of the body module; and a foot location detection module coupled to side surfaces of the support module and configured to detect the locations of the feet of the user.

The body module may adjustable in length in the longitudinal direction thereof, and may be rotatable around the longitudinal direction.

The VR chair unit may further include a movement module configured to receive a predetermined signal and to move the body module.

The support module may be located to come into contact with the abdomen of the user and also support the upper body of the user when the user sits on the saddle module.

The brainwave-connected unit may include a brainwave detection module configured to detect brainwaves of the user and a depression analysis module configured to analyze a depressive state in association with the detected brainwaves; and the depression analysis module may be previously provided with the signature of a reference brainwave associated with depression and insomnia, and may monitor the level of the relief of depression and insomnia attributable to training by comparing and analyzing the brainwaves, detected when the user does the training, and the signature of the reference brainwave.

The brainwave-connected unit may further include a motion sickness analysis module configured to analyze a motion sickness state in association with the detected brainwaves.

The kiosk may further include a physical capacity measurement module configured to measure the physical capacity of the user; and the VR glove unit and the VR chair unit may be adjustable based on the data of the physical capacity measurement module.

### BRIEF DESCRIPTION OF THE DRAWINGS

The above and other objects, features, and advantages of the present invention will be more clearly understood from the following detailed description taken in conjunction with the accompanying drawings, in which:

FIG. 1 is a conceptual diagram schematically showing a VR-based cognitive training system for relieving depression and insomnia according to the present disclosure;

FIGS. 2A and 2B show block diagrams schematically illustrating the configurations of a kiosk and a VR display unit according to the present disclosure;

FIGS. 3A and 3B show block diagrams schematically illustrating the configurations of a VR glove unit and a VR chair unit according to the present disclosure;

FIGS. 4A and 4B show block diagrams schematically illustrating the configurations of a brainwave-connected unit and a motion sickness reduction unit according to the present disclosure;

FIG. 5 is a diagram showing a VR-based cognitive training system for relieving depression and insomnia according to one embodiment of the present disclosure;

FIG. 6 shows views illustrating VR-based content according to the present disclosure;

FIG. 7 shows views illustrating the kiosk according to the present disclosure in greater detail;

FIG. 8 shows views illustrating the VR glove unit according to the present disclosure in greater detail;

FIG. 9 shows views illustrating the VR glove unit according to the present disclosure in greater detail; and

FIG. 10 shows views illustrating the VR chair unit according to the present disclosure in greater detail.

### DETAILED DESCRIPTION

While embodiments of the present disclosure will be described with reference to the accompanying drawings, this is intended to help the easy understanding of the present invention. The scope of the present invention is not limited to the embodiments, but the present invention is defined only based on the scope of the attached claims.

In the following description of the present invention, when it is determined that a detailed description of a well-known function or configuration may unnecessarily obscure the gist of the present invention, the detailed description will be omitted. Throughout the present specification, like reference symbols designate like components.

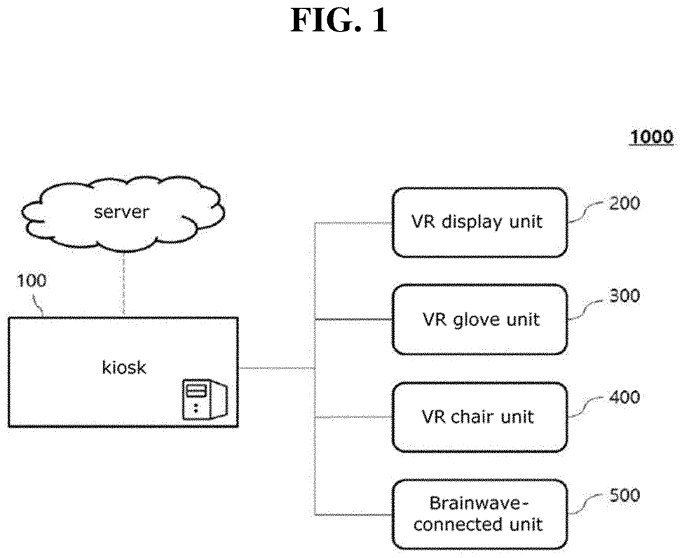

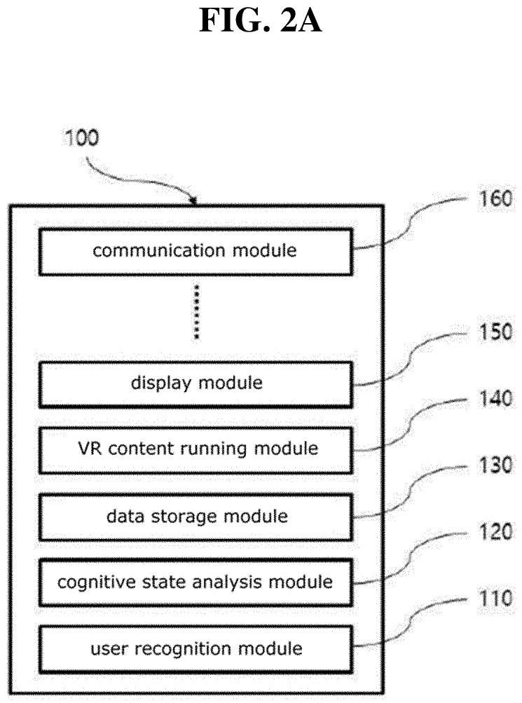

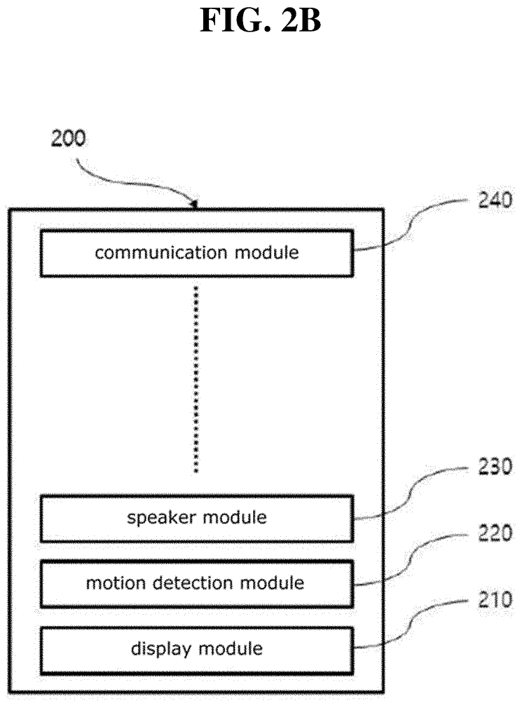

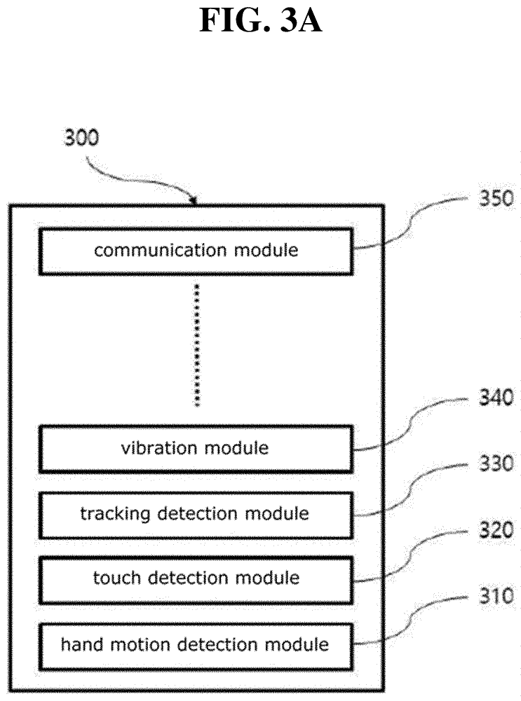

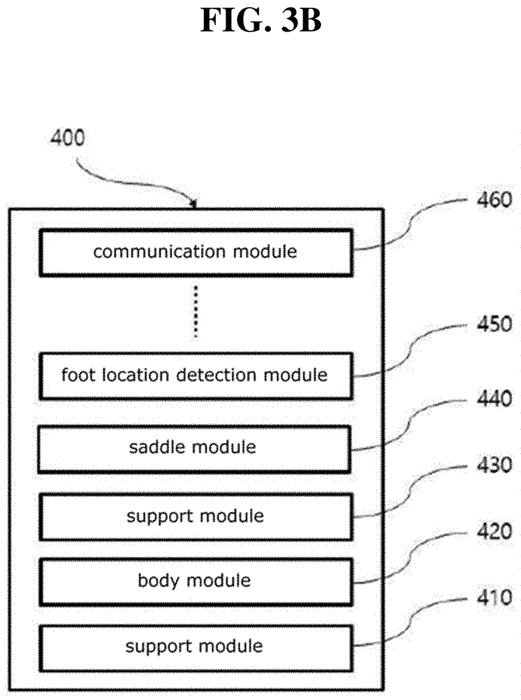

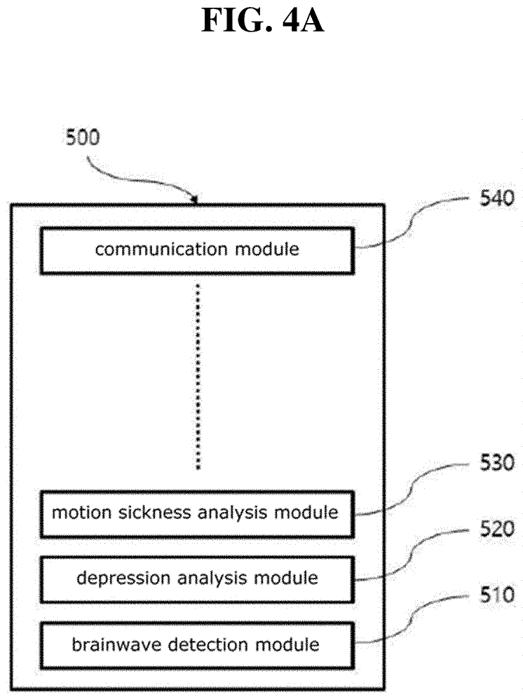

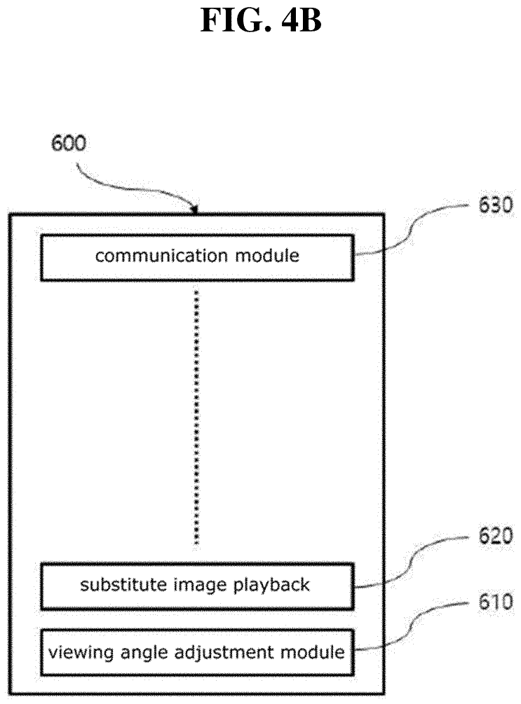

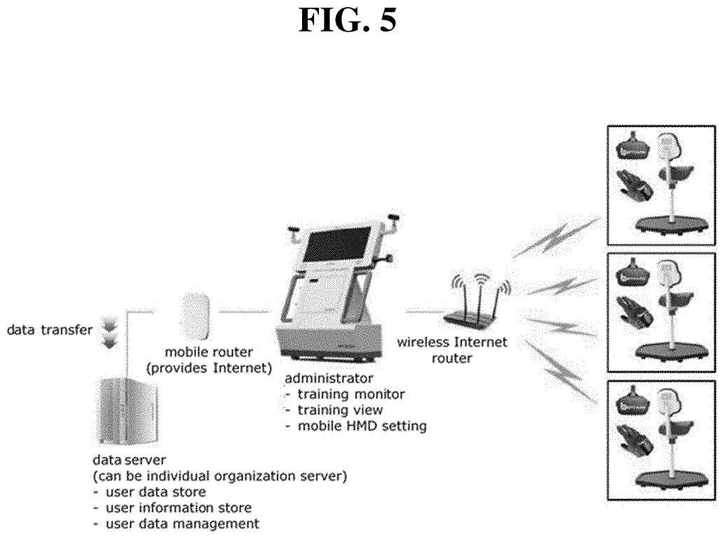

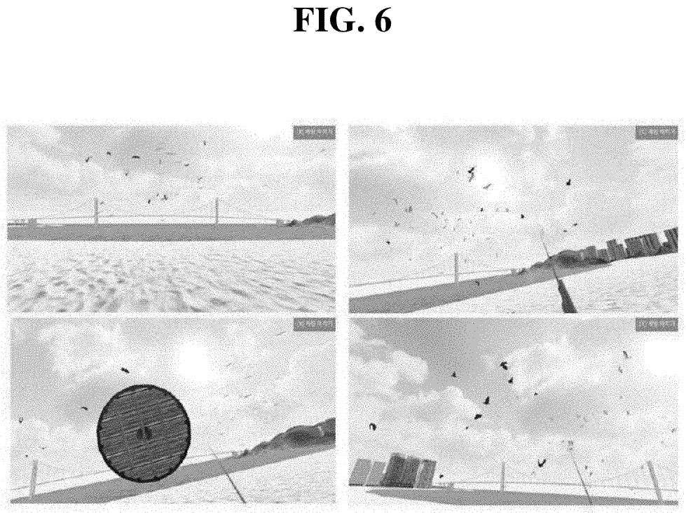

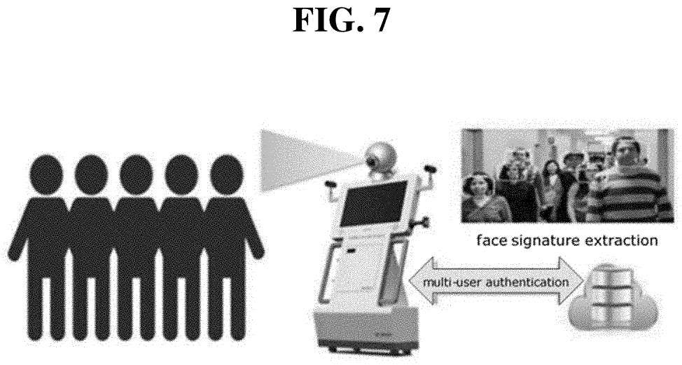

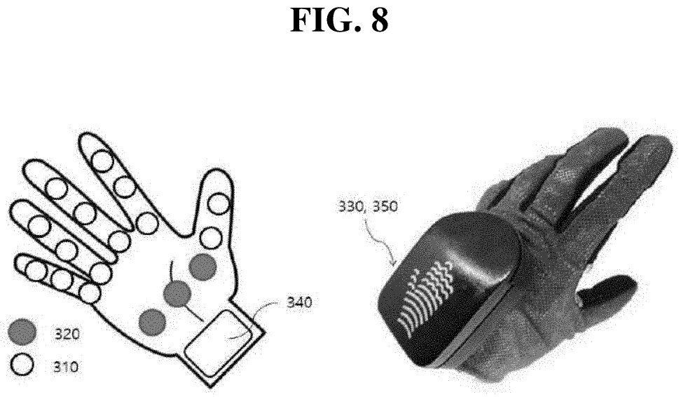

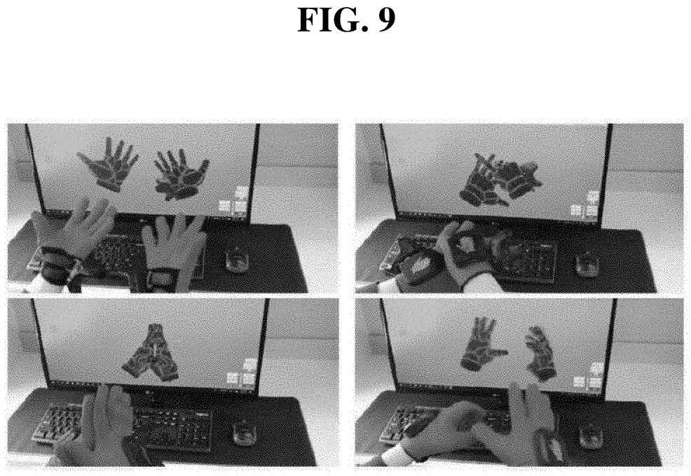

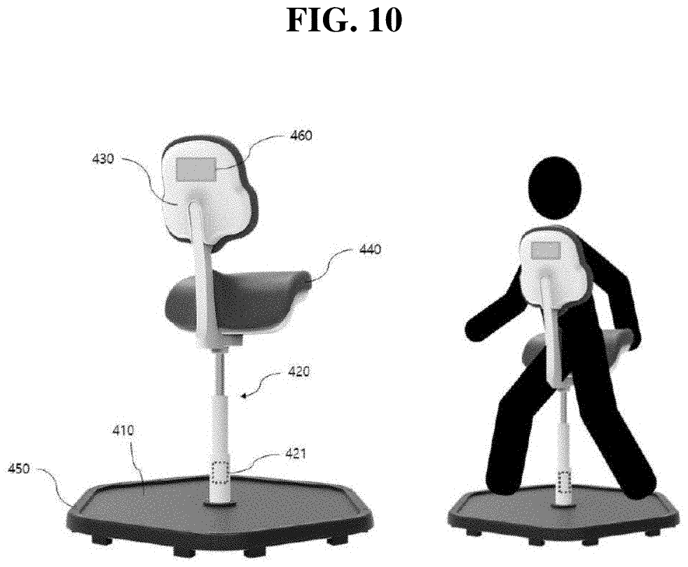

FIG. 1 is a conceptual diagram schematically showing a VR-based cognitive training system for relieving depression and insomnia according to the present disclosure, FIG. 2 shows block diagrams schematically illustrating the configurations of a kiosk 100 and a VR display unit 200 according to the present disclosure, FIGS. 3A and 3B show block diagrams schematically illustrating the configurations of a VR glove unit 300 and a VR chair unit 400 according to the present disclosure, FIGS. 4A and 4B show block diagrams schematically illustrating the configurations of a brainwave-connected unit 500 and a motion sickness reduction unit 600 according to the present disclosure, FIG. 5 is a diagram showing a VR-based cognitive training system for relieving depression and insomnia according to one embodiment of the present disclosure, FIG. 6 shows views illustrating VR-based content according to the present disclosure, FIG. 7 shows views illustrating the kiosk 100 according to the present disclosure in greater detail, FIG. 8 shows views illustrating the VR glove unit 300 according to the present disclosure in greater detail, FIG. 9 shows views illustrating the VR glove unit 300 according to the present disclosure in greater detail, and FIG. 10 shows views illustrating the VR chair unit 400 according to the present disclosure in greater detail.

<VR-Based Cognitive Training System for Relieving Depression and Insomnia>

Referring to FIGS. 1 to 5, a VR-based cognitive training system for relieving depression and insomnia according to the present disclosure may include: a kiosk 100; a VR display unit 200; a VR glove unit 300; a VR chair unit 400; and a brainwave-connected unit 500.

The kiosk 100 is configured to run VR-based content and to monitor cognitive training. As shown in FIG. 2A, the kiosk 100 may include a user recognition module 110, a data storage module 130, a VR content running module 140, a display module 150, and a communication module 160 configured to transmit and receive data.

The VR-based content is a program for cognitive training using virtual reality, and may include content for relieving depression and insomnia. The VR-based content may be game-type content that may be run in conjunction with the VR glove unit 300 and the VR chair unit 400 to be described later based on VR experience.

An example of the VR-based content will now be described with reference to FIG. 6. The content of the example is game-based content in which a plurality of objects is moving on a screen against the background of a sandy beach and a user hits the moving objects via the VR glove unit 300 and the VR chair unit 400, which will be described later.

More specifically, the content of the example is functional content in which crow-shaped objects and seagull-shaped objects are moving on a screen against the background of a sandy beach and a user blocks flying obstacles with a shield or avoids flying obstacles by moving his or her body while shooting targets, presented and selected from the crow-shaped objects and the seagull-shaped objects, with a gun, thereby performing both cognitive rehabilitation and aerobic exercise.

When doing training using the content, a user operates a gun through right hand motions via the VR glove unit 300 to be described later, operates a shield through left hand motions and performs the activities of avoiding flying and approaching obstacles through foot motions via the VR chair unit 400 to be described later, so that cognitive activities including eye, hand and foot coordination activities, attention and concentration, and decision making may be induced and stress is relieved through mild aerobic exercise effects, thereby relieving a depression and insomnia state.

Meanwhile, it will be apparent that the VR-based content is not limited to the above-described content but may include various types of content having various difficulty levels.

The user recognition module 110 may recognize a user, may register user information, and may fetch user information. The user may register information including his or her name, unique management number and photograph when using a service for the first time, and may participate in training via login.

Referring to FIG. 7, the user recognition module 110 may be configured to log in through face recognition via a camera, and may be configured to enable multi-user authentication by extracting the face signature of each user.

Meanwhile, although the user recognition module 110 has been described as performing face recognition via a camera, the user recognition module 110 is not limited thereto, but may be configured to use various methods (for example, fingerprint recognition, etc.) as desired.

The data storage module 130 may be configured to put data, received via the communication module 160, into a database and store the data. The data of the VR-based content may be stored in the data storage module 130.

A configuration may be made such that VR-based content is run by the VR content running module 140 and the run content is transmitted to the VR display unit 200 to be described later by the communication module 160 and implemented on the display module 210 of the VR display unit 200.

The VR content running module 140 may be configured to run content appropriate for a user based on a database stored in the data storage module 130.

Furthermore, the VR content running module 140 may be configured to run content having a difficulty level appropriate for a user based on a database stored in the data storage module 130.

For example, the VR content running module 140 may be configured to classify training score point ranges according to their content and difficulty level and to run content having the difficulty level of a range, within which the average score points of a user fall, based on the training database of the user.

The display module 150 may be configured to display data received via the communication module 160 and to enable a user's training to be monitored. The display module 150 may include a touch panel capable of data input, and may be configured such that a user or administrator may input data or a signal by making a touch via the display module 150.

Furthermore, the kiosk 100 may be configured to transmit the user's training data to the server of a related organization or the like via the communication module 160 and to enable the related organization to perform monitoring based on the transmitted data.

Meanwhile, the kiosk 100 may further include a cognitive state analysis module 120 configured to diagnose the degree of depression and insomnia of a user.

The cognitive state analysis module 120 may include at least one of Center for Epidemiologic Studies Depression Scale (CES-D), Beck Depression Inventory (BDI), Patient Health Questionnaire-9 (PHQ-9), Geriatric Depression Scale (GDS), Generalized Anxiety Disorder-7 (GAD-7), Rosenberg Self Esteem Scale (RSES), and Perceived Stress Scale (PSS) examination programs, and may be configured to provide analysis results based on the results of the examination.

For example, the CES-D examination may be configured to classify a state in question as a normal state when a total score is equal to or lower than 15 points, as a slight depressive state when a total score is equal to or higher than 16 points and is equal to or lower than 20 points, as a moderate depressive state when a total score is equal to or higher than 21 points and is equal to or lower than 24 points, and as a serious depressive state when a total score is equal to or higher than 25 points.

For example, the BDI examination may be configured to classify a state in question as a normal state when a total score is equal to or lower than 9 points, as a slight depressive state when a total score is equal to or higher than 10 points and is equal to or lower than 15 points, as a moderate depressive state when a total score is equal to or higher than 16 points and is equal to or lower than 22 points, and as a serious depressive state when a total score is equal to or higher than 23 points.

Meanwhile, the cognitive state analysis module 120 may be configured to transmit the examination result data to the server of the related organization via the communication module 160, to receive analysis result data from the related organization, and to provide the analysis result data to a user.

The above-described VR content running module 140 may be configured to run content having a difficulty level appropriate for a user based on the analysis result data.

Furthermore, the kiosk 100 may further include a VR content-connected module (not shown) configured to connect the data of each user when a plurality of users participates together.

The VR content-connected module (not shown) may be configured to receive the training data of at least two users, to connect data, and to transmit the connected data to the VR display units 200 of the respective users.

For example, the VR content-connected module (not shown) may be configured to connect data so that score points acquired by a plurality of users from VR-based content are connected and displayed on a single screen and to transmit the connected data to the VR display units 200 of the respective users so that the connected VR-based content can be implemented on the VR display units 200 of the respective users. As a result, a plurality of users may participate in training together, and score results based on the training are connected and displayed on a screen, thereby inducing cooperation and competition among the users and thus achieving the effect of further increasing training effects.

Furthermore, the kiosk 100 may further include a physical capacity measurement module (not shown) configured to measure a user's height, weight, hand movement-related grip force, and foot movement-related knee joint flexibility, and may be configured such that the VR-based content and the VR glove unit 300 and the VR chair unit 400 to be described later may be adjusted based on the data of the physical capacity measurement module. Although the physical capacity measurement module (not shown) has been described as being configured to measure a user's height, weight, hand movement-related grip force, and foot movement-related knee joint flexibility, it is not limited thereto.

The VR display unit 200 is configured to receive data from the above-described kiosk 100 and to visually implement VR-based content, and may include a display module 210, a motion detection module 220, a speaker module 230, and a communication module 240, as shown in FIG. 2B.

The VR display unit 200 may be connected to the above-described kiosk 100 and the VR glove unit 300, the VR chair unit 400 and the brainwave-connected unit 500 to be described later via a wired or wireless connection, and may receive and transmit signals and data via the communication module 240.

The display module 210 may be configured to receive VR-based content data from the kiosk 100 via the communication module 240 and to provide a virtual reality screen to a user.

The motion detection module 220 may be configured to detect a user's head motion and to incorporate the head motion into the VR-based content so that a virtual reality environment varies depending on the user's head motion in the content.

The motion detection module 220 may be configured to include a noise removal unit (not shown) in order to achieve more precise detection. For example, the noise removal unit (not shown) may be configured to sample data including noise at predetermined time intervals, to compare the sampled data with immediately previously sampled data, to adopt the later sampled data when the difference between the values falls within a predetermined range, and to adopt the immediately previously sampled data when the difference between the values is out of the predetermined range.

More specifically, in the case where the predetermined range is set to A, when the absolute value of the difference between the pieces of sampled data is smaller than A, the later sampled data is determined not to have noise and is adopted. In contrast, when the absolute value of the difference between the pieces of sampled data is larger than A, the later sampled data is determined to include noise and is not adopted, but the previously sampled data is adopted.

Meanwhile, the sampling time interval and the absolute value of the difference between the pieces of data may be changed. Generally, noise may be removed using a bandpass filter used in signal processing or the like.

The speaker module 230 may be configured not only to provide the sound of the content but also to receive a signal from the kiosk 100 and provide an audible notification to a user.

The VR glove unit 300 may be configured to detect a user's hand motion and to manipulate VR-based content, and may include a hand motion detection module 310, a touch detection module 320, a tracking detection module 330, and a communication module 350, as shown in FIGS. 3(a) and 8.

The VR glove unit 300 may be connected to the above-described kiosk 100 and the VR display unit 200 via a wired or wireless connection, and may transmit and receive signals and data via the communication module 350.

The hand motion detection module 310 may be installed at at least one predetermined location, and may be configured to detect and recognize a user's hand motion. The user's motion recognized by the hand motion detection module 310 is incorporated into VR-based content, as shown in FIG. 9.

The touch detection module 320 may be installed at at least one predetermined location, and may be configured to recognize touch.

The tracking detection module 330 may be configured to detect the three-dimensional (3D) trajectory of the VR glove unit 300 and to incorporate the 3D trajectory into VR-based content. The tracking detection module 330 may include an IMU sensor, and may be configured to enable direction and rotation data to be connected via the IMU sensor. More specifically, the IMU sensor may include a six-axis sensor including a gyroscope and an accelerometer, or may include a nine-axis sensor including a gyroscope, an accelerometer, and a geomagnetic sensor.

The flexion and extension data of the fingers may be measured via the detection modules 310, 320 and 330, and an operation interface using the data may be constructed.

Meanwhile, the detection modules 310, 320 and 330 may further include a noise removal unit (not shown), such as that described in conjunction with the VR display unit 200, in order to perform more accurate detection.

Furthermore, the VR glove unit 300 may further include a vibration module 340 configured to provide a vibrational tactile experience to a user in response to a predetermined signal. For example, the vibration module 340 may be configured to provide vibration when a user catches a target or hits a target and a score is changed in VR-based content.

Furthermore, the VR glove unit 300 may further include a voice recognition module (not shown), and may be configured to recognize a predetermined voice signal and to be operated. For example, a configuration may be made such that a user who is unfamiliar with hand motion may perform operation through voice recognition, by which even the user who is unfamiliar with hand motion may do training in various ways.

The VR chair unit 400 is configured to detect the foot motion of a user and to manipulate VR-based content. The VR chair unit 400 may include a support module 410, a body module 420, a support module 430, a saddle module 440, a foot location detection module 450, and a communication module 460, as shown in FIGS. 3(b) and 10.

The VR chair unit 400 may be connected to the above-described kiosk 100 and VR display unit 200 via a wired or wireless connection, and may transmit and receive signals and data via the communication module 460.

The support module 410 is located in contact with the ground surface. Although the support module 410 is preferably formed in a hexagonal plate shape, it is not limited thereto. A user steps, moves, or performs a tab operation on the support module 410.

Furthermore, the top surface of the support module 410 may be further provided with a cushion unit (not shown) made of an elastic material, thereby increasing safety during training and also minimizing noise generated during training.

Meanwhile, the bottom surface of the support module 410 may be provided with at least one height adjustment unit (not shown) configured to adjust the balance of the support module 410. The height adjustment unit (not shown) may be configured to adjust the height of the support module 410 in a vertical direction through rotation. Accordingly, the horizontal adjustment of the support module 410 may be easily performed according to the bending, inclination or the like of the bottom surface of a place where the support module 410 is located, and also the support module 410 is maintained in a stopped state, thereby providing safety during training.

Meanwhile, although the height adjustment unit (not shown) has been described as adjusting the height in a vertical direction, it will be apparent that the height adjustment unit is not limited thereto but may be configured in various ways as desired.

Furthermore, the bottom surface of the support module 410 may be provided with casters (not shown) configured to provide the convenience of movement. Although the casters (not shown) are preferably configured to be attached and then support movement when required, it will be apparent that the casters are not limited thereto but may be configured in various ways as desired.

The body module 420 may be formed to a predetermined length in an upward direction from the center of the support module 410. The body module 420 may be configured to be adjusted in length in the longitudinal direction thereof and to be rotated around the longitudinal direction.

Meanwhile, the VR chair unit 400 may further include a movement module 421 configured to be disposed inside the body module 420 and to receive a predetermined signal and adjust the length of the body module 420 or rotate the body module 420. This provides a user with an environment in which the VR chair unit 400 automatically moves according to VR-based content, thereby providing the advantage of enabling a user to perform various types of experiential training.

The support module 430 is coupled to one end of the body module 420, and may be configured to be located to come into contact with the abdomen of a user and also support the upper body of the user when the user sits on the saddle module 440 to be described later. The support module 430 is preferably configured to be adjustable in length in a vertical direction and to be applicable to users of various body sizes.

The saddle module 440 may be formed at a predetermined location of the body module 420 in a direction perpendicular to the longitudinal direction, and may be configured such that a user may sit thereon. The saddle module 440 is preferably configured to be provided to be adjustable in location in a direction perpendicular to the longitudinal direction of the body module 420 and to be applicable to users of various body sizes.

Meanwhile, the saddle module 440 may further include an inclination adjustment unit (not shown) configured to adjust the inclination of the saddle module 440. This inclination adjustment unit (not shown) may perform adjustment so that the saddle module 440 is inclined with respect to the direction in which the body module 420 is located, by which the center of gravity is adjusted such that the support module 430 naturally comes into contact with and supports the abdomen of a user when the user sits, thereby reducing the risk of getting hurt from a fall during training.

The foot location detection module 450 may be coupled to the side surfaces of the support module 410, and may be configured to detect the locations of the feet of a user. The foot location detection module 450 may include a plurality of infrared sensors. Although the foot location detection module 450 is preferably configured to surround all the side surfaces of the support module 410, it is not limited thereto. The foot location detection module 450 may enable VR-based content to be manipulated through the foot motion of a user.

For example, a configuration may be made such that movement is performed in a viewing direction within VR-based content when the foot location detection module 450 detects a user's continuous tapping operation, and thus the user is induced to take aerobic exercise having a level appropriate for the user and an intuitive content manipulation method are presented, thereby arousing interest and amusement.

Furthermore, the VR chair unit 400 may further include a vibration module (not shown) configured to provide vibrational tactile experience to a user in response to a predetermined signal. For example, the vibration module (not shown) may be installed at a predetermined location on the top surface of the saddle module 440, and may be configured to provide vibration when a user collides with a wall in VR-based content, a user departs from a path in content in which a user needs to go along a predetermined path, or the like. The vibration module (not shown) may be installed at a predetermined location on the top surface of the saddle module 440 or a predetermined location of the support module 430 in contact with the abdomen of the user, and may provide vibration to the user.

Furthermore, the VR chair unit 400 may further include a voice recognition module (not shown), and may be configured to recognize a predetermined voice signal and perform manipulation. For example, a configuration may be made such that a user who is unfamiliar with foot motion may perform operation through voice recognition, by which even the user who is unfamiliar with foot motion may perform training in various ways.

The brainwave-connected unit 500 may be configured to detect and analyze brainwaves of a user during training, and may include a brainwave detection module 510, a depression analysis module 520, and a communication module 540, as shown in FIG. 4A.

The brainwave-connected unit 500 may be connected to the above-described kiosk 100 via a wired or wireless connection, and may transmit and receive data via the communication module 540.

The brainwave detection module 510 may be configured to detect brainwaves of a user, may include a plurality of electrodes attached to different locations of the head of the user, and may be configured to receive brainwaves from the plurality of electrodes.

Although an Ag—AgCl disk capable of accurately measuring a slow change in voltage is preferably used as the electrodes used to measure brainwaves, they are not limited thereto.

Brainwaves may be detected using the electrodes. Generally, brainwaves may be classified into delta waves, theta waves, alpha waves, beta waves, and gamma waves according to their frequency. Delta waves are signals with a frequency of 0.2 to 4 Hz, theta waves are signals with a frequency of 4 to 8 Hz, alpha waves are signals with a frequency of 8 to 12 Hz, beta waves are signals with a frequency of 12 to 30 Hz, and gamma waves are signals with a frequency above 30 Hz.

More specifically, delta waves are brainwaves that are observed when people are in deep sleep, and theta waves are brainwaves that are generated when people utilize information inside their brains or concentrate on the solution of a logical thinking problem. Furthermore, alpha waves are brainwaves that are generated when people concentrate their minds and utilize information inside their brains, beta waves are brainwaves that are generated mainly when they are physically active or are immersed in something, and gamma waves are brainwaves that are generated when a nervous and active complex mental function is performed.

Meanwhile, depression and insomnia are particularly related to the alpha waves of brainwaves. Accordingly, brainwaves in the alpha wave range are extracted from the received brainwaves, a power spectrum is measured, and the measured power spectrum is subjected to Fast Fourier Transform (FFT), thereby calculating power spectrum density.

Although it is preferable that the brainwave detection module 510 include a data preprocessing unit (not shown) configured to perform noise removal, signal amplification and the like in order to achieve more accurate detection and use an Emotive Epoc+ device as a data collection device, the brainwave detection module 510 is not limited thereto.

The depression analysis module 520 may be configured to receive brainwave data from the brainwave detection module 510 and determine whether or not a user is in a depressive state. This depression analysis module 520 may be previously provided with the signature of a reference brainwave associated with depression and insomnia. The depression analysis module 520 may be configured to enable a user to compare brainwaves detected during training with the reference brainwave and determine the level of the relief of depression and insomnia attributable to training.

Meanwhile, the alpha waves of brainwaves are also associated with motion sickness, and thus the brainwave-connected unit 500 may further include a motion sickness analysis module 530 configured to receive brainwave data from the brainwave detection module 510 and to detect and analyze the motion sickness state of a user. This motion sickness analysis module 530 may be previously provided with a reference value for each user in order to determine whether or not a user is in a motion sickness state.

For example, the motion sickness analysis module 530 may set baseline power for each user so that the alpha waves generated when the user is using VR-based content may be compared with the alpha waves generated before the user uses the VR-based content. More specifically, the motion sickness analysis module 530 may set the baseline power by using brainwaves of the user measured in a state in which a preset reference image is output. This reference image refers to an image in which the user cannot feel motion sickness, which may be a still image or a fixed image.

In other words, the motion sickness analysis module 530 may be configured to receive data from the brainwave detection module 510, to calculate power variation by comparing the received data with the previously measured baseline power, and to determine whether or not a user is in a motion sickness state by comparing the calculated power variation with a predetermined critical range.

Meanwhile, in connection with the motion sickness analysis module 530, the VR-based cognitive training system for relieving depression and insomnia according to an embodiment of the present disclosure may further include a motion sickness reduction unit 600 configured to reduce the motion sickness state of a user. The motion sickness reduction unit 600 may include a viewing angle adjustment module 610, a substitute image playback module 620, and a communication module 630, as shown in FIG. 4B.

The motion sickness reduction unit 600 may be connected to the above-described kiosk 100, VR display unit 200 and brainwave-connected unit 500 via a wireless or wired connection, and may transmit and receive data via the communication module 630.

The viewing angle adjustment module 610 may be configured to adjust the viewing angle of VR-based content implemented in the VR display unit 200 in order to relieve the motion sickness state when it is determined by the motion sickness analysis module 530 that a user is in a motion sickness state. For example, a configuration may be made to relieve motion sickness by limiting the viewing angle of VR-based content implemented in the VR display unit 200.

The substitute image playback module 620 may include a camera unit (not shown) connected to the above-described VR display unit 200 and configured to photograph the outside. The camera unit (not shown) may be connected to the outer circumferential surface of the VR display unit 200 in order to photograph the viewing direction of a user when the user wears the VR display unit 200. Accordingly, the camera unit (not shown) may be configured to take an image identical to that viewed by the user without wearing the VR display unit 200.

Meanwhile, the substitute image playback module 620 may be configured to play back an image, photographed by the camera unit (not shown), in place of VR-based content on the VR display unit 200 when the motion sickness state of a user is not relieved even by the above-described viewing angle adjustment module 610. Accordingly, the user may rapidly relieve the motion sickness state by using the effect of feeling as if the user did not wear the VR display unit 200.

Accordingly, the VR-based cognitive training system for relieving depression and insomnia according to an embodiment of the present disclosure has an advantage in that a user may do training at a desired time and place using a virtual reality program.

Furthermore, the VR-based cognitive training system for relieving depression and insomnia according to an embodiment of the present disclosure has the advantage of efficiently receiving training at a relatively low cost compared to attending a hospital for the treatment of depression and insomnia.

Furthermore, the VR-based cognitive training system for relieving depression and insomnia according to an embodiment of the present disclosure has the advantage of increasing training effects by improving the degree of immersion in training in such a manner as to arouse interest and amusement using VR-based content.

Furthermore, the VR-based cognitive training system for relieving depression and insomnia according to an embodiment of the present disclosure has an advantage in that a plurality of participants may use a program together, so that concentration may be intensified and the participants may be instilled with feelings of cooperation, accomplishment and belonging compared to conventional solo training, thereby improving training effects.

Furthermore, the VR-based cognitive training system for relieving depression and insomnia according to an embodiment of the present disclosure has the advantage of monitoring training effects in real time via the brainwave measurement unit configured to collect and analyze brainwaves.

Furthermore, the VR-based cognitive training system for relieving depression and insomnia according to an embodiment of the present disclosure has the advantage of reducing cyber motion sickness attributable to the use of a VR apparatus by detecting and dealing with the motion sickness in such a manner as to collect and analyze specific brainwaves.

Moreover, the VR-based cognitive training system for relieving depression and insomnia according to an embodiment of the present disclosure has the advantage of doing training through 3D motions even within a limited space via the VR chair unit.

While the embodiments of the present disclosure have been described with reference to the accompanying drawings, it will be apparent to those of ordinary skill in the art to which the present invention pertains that various modifications and alterations may be made based on the foregoing description within the scope of the present invention.

What is claimed is:

1. A virtual reality (VR)-based cognitive training system for relieving depression and insomnia, the VR-based cognitive training system comprising:
a kiosk configured to run VR-based content for relieving depression and insomnia;
a VR display unit configured to receive data from the kiosk and to visually implement the VR-based content;
a VR glove unit configured to detect a hand motion of a user and to manipulate the VR-based content;
a VR chair unit configured to detect a foot motion of a user and to manipulate the VR-based content; and
a brainwave-connected unit configured to detect and analyze brainwaves of the user,
wherein when the user wears the VR display unit and the VR glove unit, sits on the VR chair unit and does training according to the VR-based content, the brainwave-connected unit detects and analyzes brainwaves of the user during the training, and the kiosk monitors effects of the training.

2. The VR-based cognitive training system of claim 1, wherein the VR glove unit comprises:
a hand motion detection module installed at at least one predetermined location and configured to detect a hand motion of the user;
a touch detection module installed at at least one predetermined location and configured to recognize a touch; and
a tracking detection module configured to detect a trajectory of the VR glove unit.

3. The VR-based cognitive training system of claim 2, wherein the VR chair unit comprises:
a support module configured to be disposed on a ground surface and to form a support;
a body module formed to a predetermined length in an upward direction from a center of the support module;
a support module coupled to one end of the body module;
a saddle module formed at a predetermined location of the body module in a direction perpendicular to a longitudinal direction of the body module; and
a foot location detection module coupled to side surfaces of the support module and configured to detect locations of feet of the user.

4. The VR-based cognitive training system of claim 3, wherein the body module is adjustable in length in the longitudinal direction thereof, and is rotatable around the longitudinal direction.

5. The VR-based cognitive training system of claim 4, wherein the VR chair unit further comprises a movement module configured to receive a predetermined signal and to move the body module.

6. The VR-based cognitive training system of claim 3, wherein the support module is located to come into contact with an abdomen of the user and also supports an upper body of the user when the user sits on the saddle module.

7. The VR-based cognitive training system of claim 1, wherein the brainwave-connected unit comprises a brainwave detection module configured to detect brainwaves of the user and a depression analysis module configured to analyze a depressive state in association with the detected brainwaves, and
wherein the depression analysis module is previously provided with a signature of a reference brainwave associated with depression and insomnia, and monitors a level of relief of depression and insomnia attributable to training by comparing and analyzing the brainwaves, detected when the user does the training, and the signature of the reference brainwave.

8. The VR-based cognitive training system of claim 7, wherein the brainwave-connected unit further comprises a motion sickness analysis module configured to analyze a motion sickness state in association with the detected brainwaves.

9. The VR-based cognitive training system of claim 8, wherein the kiosk further comprises a physical capacity measurement module configured to measure a physical capacity of the user, and
wherein the VR glove unit and the VR chair unit are adjustable based on data of the physical capacity measurement module.
